(19) United States
(12) Patent Application Publication
(10) Pub. No.: US 2009/0105532 A1
(43) Pub. Date: Apr. 23, 2009

(54) In vivo imaging device and method of manufacturing thereof

(76) Inventor: Zvika Gilad, Hod Hacarmel (IL)

Correspondence Address: Pearl Cohen Zedek Latzer, LLP, 1500 Broadway, 12th Floor, New York, NY, 10036, US

(21) Appl. No.: 11/976,106

(22) Filed: Oct. 22, 2007

(51) Int. Cl. A61B 1/00

(52) U.S. Cl. 600/101

(57) ABSTRACT

A method of manufacturing an in vivo imaging device comprising the steps of: coating an electrical component with a scaffold micro-structure; assembling the micro-structure on a circuit board; and encapsulating the circuit board in a swallowable capsule.

### FIELD OF THE INVENTION

The present invention relates to an in-vivo device, system and method such as for imaging an in-vivo lumen; more specifically, to a method of manufacturing an in-vivo imaging device and to a device thus manufactured.

### BACKGROUND OF THE INVENTION

Known devices may be helpful in providing in-vivo sensing, such as imaging or pH sensing. Autonomous in-vivo sensing devices, such as swallowable or ingestible capsules or other devices may move through a body lumen, sensing as they move along. An autonomous in-vivo sensing device such as an imaging device may include, for example, an imager for obtaining images from inside a body cavity or lumen, such as the gastrointestinal (GI) tract. The imager may, for is example, be associated with an optical system, and optionally a transmitter and an antenna. Illumination units, such as LEDs, may be used to illuminate the in-vivo lumen.

Different methods of manufacturing sensitive electrical components, for example antennas or illumination units for in vivo imaging devices, are known in the art. For example, antennas may be embedded into a circuit board of the in-vivo imaging device. Such implementations may create electromagnetic interference between the antennas and/or between other electrical components or wires on the circuit board. Such interferences may degrade the function of the antenna. In another example, the antennas may be arranged around the periphery of a circuit board in the in vivo imaging device, or mounted in a flat manner over the surface of a support unit. The antennas may be made from a soft conductive material, such as copper, which may require a delicate assembly or integration procedure due to its malleable nature and the limited space in the in vivo imaging device. In another example, LEDs may be assembled on a substrate or on a circuit board of the in-vivo imaging device. The assembly process of the LEDs may not be precise and may create significant variations in the illumination capabilities of different imaging devices, for example in-vivo imaging capsules.

### SUMMARY OF THE INVENTION

The present invention aims to provide a device and method of manufacture that may be easier to assemble compared to known methods.

### BRIEF DESCRIPTION OF THE DRAWINGS

The present invention will be understood and appreciated more fully from the following detailed description taken in conjunction with the drawings in which:

FIG. 1 schematically illustrates an in-vivo imaging system, according to one embodiment of the present invention;

FIG. 2 schematically illustrates an in vivo imaging device according to another embodiment of the invention;

FIG. 3 shows a diagram of an antenna embedded within a micro-structure, according to one embodiment of the present invention;

FIGS. 4A, 4B, 4C and 4D show different views of an illumination unit embedded within a micro-structure, according to one embodiment of the present invention; and

FIG. 5 shows a flow chart depicting a method of manufacture of an in vivo imaging device, according to an embodiment of the present invention.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE INVENTION

In the following description, various aspects of the present invention will be described. For purposes of explanation, specific configurations and details are set forth in order to provide a thorough understanding of the present invention. However, it will also be apparent to one skilled in the art that the present invention may be practiced without the specific details presented herein. Furthermore, well known features may be omitted or simplified in order not to obscure the present invention.

Embodiments of the system and method of the present invention may be used in conjunction with an imaging system or device such as embodiments described in U.S. Pat. No. 7,009,634 to Iddan et al. entitled A DEVICE AND SYSTEM FOR IN VIVO IMAGING. However, the device, system and method according to the present invention may be used with any suitable device, system and method providing imaging and other data from a body lumen or cavity. One embodiment of the device and system of the present invention may include an imaging device, which may be, for example, a capsule, for example, particularly suited for imaging the gastrointestinal tract or other lumens, although of course other suitable portions of the body may be imaged. Embodiments of the present invention may be incorporated into or used with other imaging capsules or devices, having other structures.

Miniaturized components may be used in imaging capsules or devices, since such devices may be limited in size (given that, for example, they should be easily swallowed by the patient, and/or as they may need to pass through relatively small apertures). Production of a reduced size imaging device may be achieved by producing electrical components embedded in three-dimensional micro-structures, for example in the micrometer or nanometer range, by using known techniques, such as Rapid Micro Product Development (RMPD™), a generative production method developed by the German company microTEC. With RMPD™ technology miniaturized components may be “grown” according to required specifications by layering a material such as molten plastic.

Embodiments of the device may comprise a capsule or other unit where all the components are substantially contained within a container or shell, and where the device does not require any wires or cables to, for example, receive power or transmit information. The device may communicate with an external receiving and display system to provide display of data, control, or other functions. For example, power may be provided by an internal battery or a wireless receiving system. Other embodiments may have other configurations and capabilities. For example, components may be distributed over multiple sites or units. Control information may be received from an external source.

The present invention provides an in vivo imaging device (such as a swallowable imaging capsule) having a sensitive or malleable component in a rigid covering, to allow easy assembly of the component in the in vivo imaging device, for example a component such as an antenna or illumination unit. The manufacturing process may be performed in such a manner that the micro-structure is built up around an individual electronic component or multiple electronic components. In some embodiments, the aid of tooling may not be required in the manufacturing process. Other manufacturing methods may be used, for example a plastic component may be manufactured using injection molding techniques, wherein molten plastic is injected at high pressure into a mold. The plastic component may be created with cavities or apertures for insertion of, for example, a metallic component into the plastic. The plastic component may be metal-plated, e.g., a thin film metal may be formed onto it, and the required pattern of the metal may be created by for example laser removal of redundant material around the pattern.

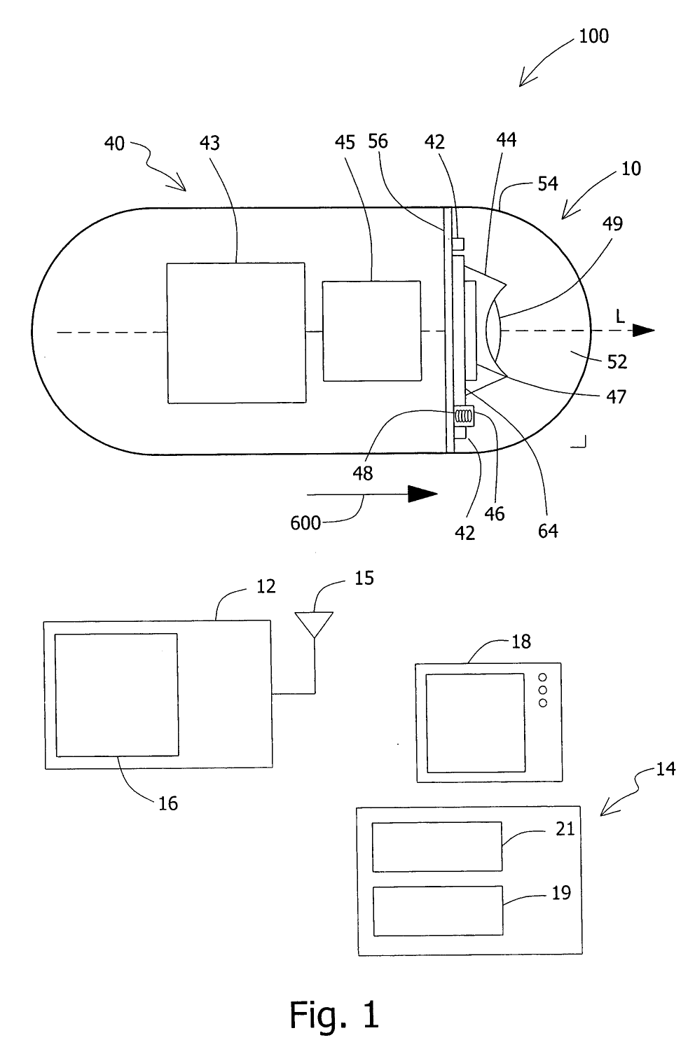

Reference is made to FIG. 1, which shows a schematic diagram of an in-vivo imaging system 100 according to one embodiment of the present invention. The in-vivo imaging system 100 may include, for example, an in-vivo imaging device 40. The in-vivo device 40 may be, for example, a swallowable capsule capturing images and possibly other data. The in-vivo device 40 may be in the shape of a capsule, including for example a viewing window or dome 54; other shapes may be used, and the device need not be swallowable or a capsule. Typically, device 40 may include an optical system 10 including, for example, one or more lens(es) 49, lens holder, baffle, or separator 44, a sensor such as an imager 47, for capturing images, and a processing chip or circuit that processes the signals generated by the imager 47. A processing circuit need not be a separate component; for example, processor or a processing chip may be integral to the imager 47. The processing circuit may be divided into several different units or separate components. An illumination source(s) 42, such as a set of light emitting diodes (LEDs), organic LEDs (OLEDs), or other suitable light sources, may provide light to illuminate objects.

According to one embodiment of the present invention, the device 40 typically may include an imager 47, which may be a complementary metal oxide semiconductor (CMOS) imaging camera. The CMOS imager is typically an ultra low power imager and is provided in chip scale packaging (CSP). Other types of CMOS imagers may be used. In another embodiment, another imager may be used, such as a CCD imager, or another imager. According to other embodiments a 320×320 pixel imager may be used. Pixel size may be between 5 to 6 micron. Other pixel sizes may be used. According to some embodiments pixels may be each fitted with a micro lens.

The device 40 typically may include a transmitter/receiver 43, for transmitting and/or receiving image and other (e.g., non-image) information to a receiving device, and may include other components. The transmitter/receiver 43 may be an ultra low power radio frequency (RF) transmitter with high bandwidth input, possibly provided in chip scale packaging, and may be combined with processing chip or circuit. The transmitter/receiver 43 may transmit and/or receive via for example an antenna 48. The transmitter/receiver 43 may also act as a controller and include circuitry and functionality for controlling the device 40, although a separate control unit may be used.

Antenna 48 may be comprised of a conducting material, such as copper, and may be held within a micro-structure supporting unit, for example micro-structure 46. Typically, the antenna 48 may be made of a malleable material that may easily bend or become deformed, for example during the assembly process or when exposed to excessive heat. Micro-structure 46 may be created by layering a non-conducting material, e.g. molten plastic, in such manner that surrounds or partially coats the antenna 48. The micro-structure 46 may be made of a material that is non-flexible, such as acrylate to provide support to the antenna. Other materials may be used to create the micro-structure and cover the antenna, preferably non-conducting materials that may provide insulation as well as support for the antenna. Micro-structure 46 may be fixed or otherwise attached to a substrate such as, for example, circuit board 64 or directly positioned onto a substrate 56. In other embodiments, circuit board 64 may be further attached to a substrate 56, which may for example support illumination source(s) 42. Illumination source(s) 42 may be embedded in a separate micro-structure, and may be attached to or embedded in micro-structure 46. Illumination source(s) 42 may be supported by an independent scaffold, substrate or circuit board, which may be supported by or integrated with substrate 56 and which may define a viewing direction 60 of device 40. Substrate 56 may be for example a rigid circuit board or a rigid-flex circuit board. In other embodiments, the illumination source(s) may be positioned on a different plane than, for example, imager 47.

Typically, the device may include a power source 45, such as one or more batteries. For example, the power source 45 may include silver oxide batteries, lithium batteries, or other electrochemical cells having a high energy density, or the like. Other power sources may be used.

Other components and sets of components may be used. For example, the power source may be capable of receiving power from an external power source transmitting power to the device 40, and a controller separate from the transmitter/receiver 43 may be used.

Preferably, located outside the patient's body in one or more locations, external to the in-vivo device 40, are a receiver 12, preferably including an antenna or antenna array 15, for receiving image and possibly other data from device 40, a receiver storage unit 16, for storing image and other data, a data processor 14, a data processor storage unit 19, and an image monitor 18, for displaying, inter alia, the images transmitted by the device 40 and recorded by the receiver 12. Typically, the receiver 12 and receiver storage unit 16 are small and portable, and are worn on the patient's body during recording of the images. According to some embodiments, data processor 14, data processor storage unit 19 and monitor 18 are part of a personal computer or workstation, which may include components such as a processor or controller 21, a memory (e.g., storage 19, or other memory), a disk drive, and input-output devices, although alternate configurations are possible. In alternate embodiments, the data reception and storage components may be of another configuration. In addition, a data decompression module for decompressing data may also be included.

According to some embodiments of the present invention, a device such as device 40 may include a distance parameter measurement unit which may include an energy emitting unit or source, such as a dedicated collimated irradiation source 11. In some embodiments the irradiation source 11 may be provided for example, to measure and/or enable determination of the size of an in-vivo object and/or the distance of the in-vivo object from an in-vivo device, such as device 40. The irradiation source may reside externally to the device body, for example, in an extra-body unit. Other components or sets of components may be used. The irradiation source may include, for example, laser diodes, regular lenses and/or micro-lenses which may be attached to diodes/detectors, and may be embedded into a micro-structure according to one embodiment of the present invention, for example in addition to or instead of illumination units which may also reside in the same micro-structure or in separate micro-structures.

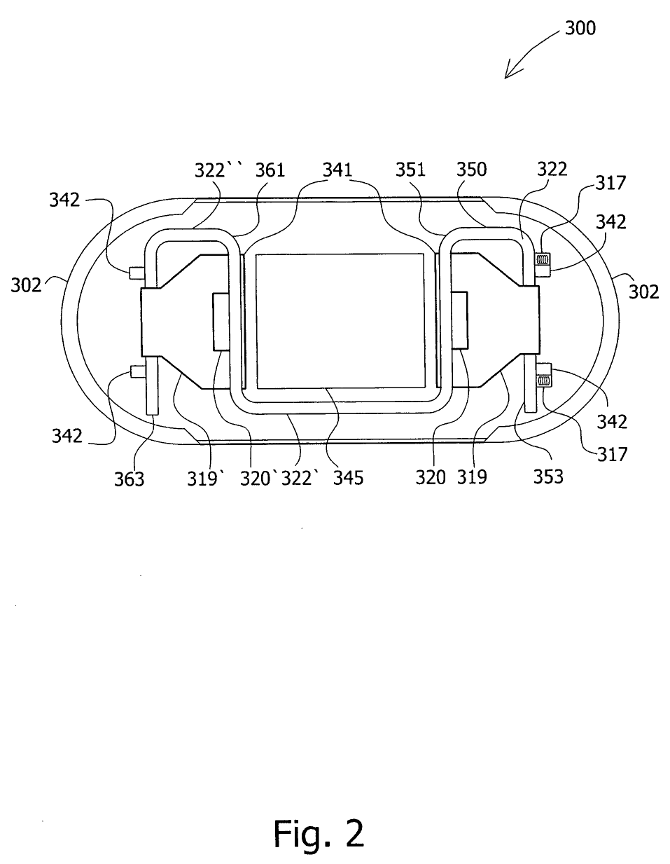

Another embodiment of the invention is schematically illustrated in FIG. 2, wherein a longitudinal cross section of device 300 is schematically shown. According to one embodiment of the present invention, device 300 may include two optical domes 302 behind which are situated illumination sources 342, two lens holder 319 and 319′, two imagers 320 and 320′ a transmitter such as an ASIC and a processor. The device 300 may further include a power source 345, which may provide power to the entirety of electrical elements of the device and an antenna 317 for transmitting video signals from the imagers 320 and 320′. According to some embodiments of the present invention, the antenna 317 may be combined with, embedded within, substantially within, or attached to elements, such as a support e.g. the lens holder 319 so as to not take up a large amount of space. The antenna 317 may also be surrounded by or nestled within components such as a support, separation or isolation elements, for example by layering non-conductive material to envelope or to partially coat the conductive component. In some embodiments, the component may be covered completely and only its pads or connectors may protrude from the enveloping material. According to some embodiments of the present invention, device 300 is capable of simultaneously obtaining images of the body lumen, for example, the GI tract, from two ends of the device. For example, device 300 may be a cylindrical capsule having a front end and a rear end, which is capable of passing the entire GI tract. The system in a cylindrical capsule can image the GI tract in the front and in the rear of the capsule.

According to one embodiment of the present invention, the various components of the device 300 may be disposed on a circuit board 350 including rigid and flexible portions; preferably the components are arranged in a stacked vertical fashion. For example, rigid portion 351 of the circuit board 350 may hold a transmitter, an imager 320 and a lens holder 319, while rigid portion 361 may hold a processor, an imager 320′ and a lens holder 319′; the other side of the rigid portions 351 and 361 may include, for example, a contact 341 for battery or power source 345. According to one embodiment of the present invention, rigid portions 353 and 363 of the circuit board 350 may include, for example, an antenna 317, and an illumination source, such as one or more LEDs 342 or other illumination sources. Antenna 317 may be located on flexible portions of the circuit board 350, or on other rigid portions such as 351, 361, or 363. According to some embodiments of the present invention, each rigid portion of the circuit board may be connected to another rigid portion of the circuit board by a flexible connector portion (e.g. 322, 322′ and 322″) of the circuit board 350. According to one embodiment of the present invention, each rigid portion of the circuit board may include two rigid sections; sandwiched between the rigid sections is a flexible connector portion of the circuit board for connecting the rigid boards. In alternate embodiments, other arrangements of components may be placed on a circuit board having rigid portions connected by flexible portions.

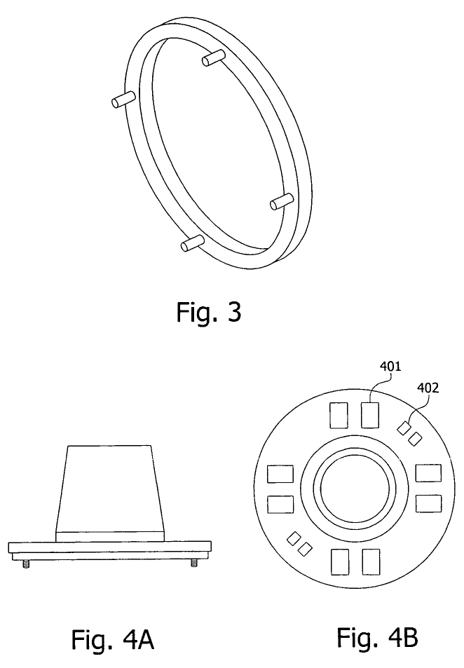

FIG. 3 depicts a schematic diagram of a plastic micro-structure, such as a scaffold, which may be created using RMPD™ technology. According to some embodiments, after the introduction of the electronic component, for example a copper antenna, layered construction of the scaffold body is performed, and a micro-structure of for example electrically (and/or thermally) non-conductive material may be built up. The non-conductive or insulating material may be layered, rising in a vertical direction, above the contacts (PADS) of the electronic component, and may construct a covering or coating that may prevent direct connection with other electronic components disposed near the antenna. However, the contacts of the coated electronic component may remain uncoated, for example in order to enable connection to an electronic circuit board, or to enable connection to another component.

A component of an in vivo imaging device, for example an antenna of a swallowable capsule, may be completely or partially coated in one or more layers, for example up to 1 ρm in thickness. According to some embodiments, the component size may be smaller than the dimensions of 1×10×10 μm. Larger component dimensions may be possible. During the layer growth process, several mechanical and/or electrical parts may be inserted to achieve the desired functionality. Other components, such as micro-optics and micro-electronics, for example an illumination source or an irradiation source, may be integrated during the manufacturing process. A micro-structured plastic scaffold may be produced, for example holding an embedded antenna within it. The plastic scaffold may contain additional components of the in vivo imaging device, for example, illumination units such as LEDs, or radiation sources such as laser diodes.

The general procedure for mechanical and electrical connecting of system components by layer-wise solidification of a liquid, light-hardenable plastic is already well known from DE4420996 entitled “Mfg. micro-mechanical and micro-optical components”, published Jan. 11, 1996 to Reiner Goetzen. Goetzen's invention concerns a procedure for manufacturing micro-mechanical and micro-optical components and complex micro-systems, by a method in which a drop of photo-induced hardenable liquid is put between two flat parallel plates, which are drawn apart in growth steps. Layers formed are shaped by guided and focused electromagnetic waves in accordance with a layered 3D volumetric model produced on a computer. The three-dimensional micro-structure does not adhere to the top plate, which is transparent to the electromagnetic waves, and fills out the cavity with the liquid material in a layering step, according to desired layered height.

An enhanced method is described in DE 19539039 C2, entitled “Improved manufacture of micro-mechanical and micro optical devices”, and published Nov. 11, 1999 to Reiner Goetzen. The invention describes using divided-up electromagnetic waves rather than lasers in all the rapid prototyping processes used to manufacture microelectronic circuits by hardening and structuring a liquid.

Materials used for RMPD™ may be plastic-type materials, for example acrylates (such as Polymethyl methacrylate—PMMA) or epoxies. According to some embodiments of the present invention, such materials should be electrically non-conductive. Other components and/or materials, for example metal and ceramic parts, may be produced in the combination of RMPD™ with other technologies, such as metal injection molding technologies.

In one embodiment of the present invention, the layered micro-structure may be grown around more than a single component, for example integrated circuit components may be embedded into a single micro-structure element. In this embodiment, the method used for creating the micro-structure may allow for example embedding an illumination source or sources, and/or an irradiation source. For example, a method as described in DE19826971C2 by Reiner Goetzen et al., entitled “Mechanical and electric coupling integrated circuits”, and published Mar. 14, 2005, concerns a procedure for mechanical and electrical connection of system component parts like integrated circuits (ICs) and further active/passive electronic as well as mechanical system component parts for the production a complex electronic, electro-optical, electro-acoustic or electro-mechanical system by layer-wise solidification of a liquid, light-hardenable plastic, whereby during the layer-wise fabrication of the module recesses are generated for the admission of the system component parts as well as linking channels for the admission of electrically conducting connections between the embedded system component parts. The existing base module is built up layer-wise, and at least one recess for the admission of one or several components may be created, while at the same time necessary linking channels are generated. After inserting the appropriate components into the recess/es, the module is further built up layer-wise, in a manner such that the recesses are locked and the components are embedded within.

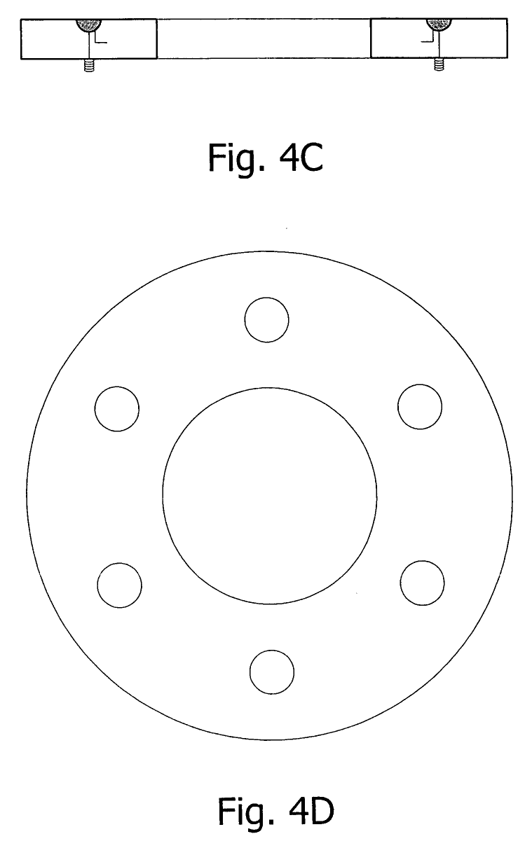

Reference is now made to FIGS. 4A and 4B, which demonstrate an example of a micro-structure which may be manufactured according to one embodiment of the invention. FIG. 4A illustrates a side view of a micro-structure which may include for example several spring-like contacts, which may protrude from the micro-structure and may be connected to a substrate and situated in the swallowable imaging device. The micro-structure may include several optical elements, such as a lens holder, and/or a spacing element, and may include contacts, for example for illumination LEDs. The micro-structure may include the illumination units, as a single component which may be assembled onto a substrate. The micro-structure may be manufactured as a single unit, for example by using the RMPD manufacturing method. According to some embodiments, the illumination units and/or the irradiation units may be manufactured as separate components, and may be assembled on the micro-structure, for example by connecting the units onto electrical contacts embedded in the micro-structure. FIG. 4B shows a bottom view of the micro-structure which may include for example electrical contacts for components such as illumination units and/or irradiation sources. According to some embodiments, the electrical components, for example LED dies which may be used as the illumination units, may also be embedded directly into the micro-structure during the manufacturing process. Such a micro-structure is illustrated in FIG. 4C, which shows a top view of a ring micro-structure with 6 embedded LED dies, and in FIG. 4D, which shows a slice view of a ring micro-structure with embedded LED dies and their suitable electrically conducting connections that connect them to an energy source. The electrically conducting connections can be produced, for example as described above, by generating recesses the admission of linking channels during the layer-wise fabrication of the module.

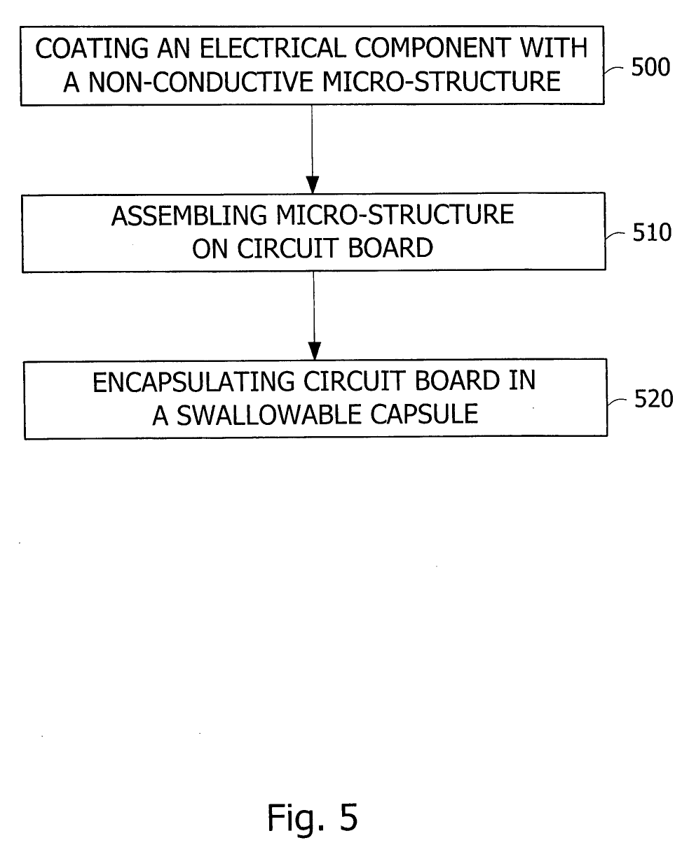

Reference is now made to FIG. 5, which illustrates a method of manufacturing an in vivo imaging device according to an embodiment of the present invention. In step 500, an electrical component, for example an irradiation source, may be coated with a non-conductive material, and a micro-structure may be created as a result of the layering (coating) process. In some embodiments, layers of conductive material may be used, and in other embodiments layers of both conductive and non-conductive materials may be used. In step 510, the micro-structure may be assembled on a circuit board, for example if the micro-structure covers an antenna, the antenna's contacts may remain uncoated, and may be connected to suitable pads prepared on the circuit board. In another embodiment, an illumination source and related optic elements may be coated by a rigid micro-structure, and may be assembled on a circuit board which may provide connection to a controller and a power source. In step 520 the circuit board may be encapsulated in a swallowable capsule. Other steps or series of steps may be used.

It will be appreciated by persons skilled in the art that the present invention is not limited to what has been particularly shown and described hereinabove. Rather the scope of the present invention is defined only by the claims, which follow:

What is claimed is:

1. A swallowable in-vivo imaging device comprising:
a circuit board;
an electronic component comprising a connecting unit, said connecting unit connectable to said circuit board; and
a micro-structure to support said electronic component, said micro-structure created by layering material over the electronic component.

2. The device of claim 1 wherein the electronic component is an antenna, an illumination unit, an irradiation unit or an integrated circuit component.

3. The device of claim 1 wherein a plurality of components are embedded into the micro-structure.

4. The device of claim 1 wherein the material is non-conductive.

5. A method of manufacturing an in-vivo imaging device comprising the steps of:
coating a component of the in-vivo imaging device with a scaffold micro-structure;
assembling the micro-structure on a circuit board; and
encapsulating the circuit board in a swallowable capsule.

6. The method of claim 5 wherein the component is partially coated by the micro-structure.

7. The method of claim 5 wherein the component is an electronic component.

8. The method of claim 7 wherein the electronic component includes electronic connectors, said connectors protruding from the micro-structure.

9. The method of claim 5 wherein a plurality of components are coated by the micro-structure.
